(19) United States
(12) Patent Application Publication
(10) Pub. No.: US 2025/0082427 A1
(43) Pub. Date: Mar. 13, 2025

(54) SYSTEMS AND DEVICES FOR ACCESSING SPACE BETWEEN TISSUE LAYERS

(71) Applicant: EDWARDS LIFESCIENCES CORPORATION, Irvine, CA (US)

(72) Inventors: Ralph Schneider, Fallbrook, CA (US); Amir Yousef Sajjadi, Aliso Viejo, CA (US); Yuval Kasher, Kfar Shmuel (IL)

(21) Appl. No.: 18/957,497

(22) Filed: Nov. 22, 2024

Related U.S. Application Data
(63) Continuation of application No. PCT/US2023/023878, filed on May 30, 2023.
(60) Provisional application No. 63/481,099, filed on Jan. 23, 2023.
(60) Provisional application No. 63/347,466, filed on May 31, 2022.

(51) Int. Cl. A61B 90/00; A61M 25/06

(52) U.S. Cl. CPC A61B 90/02; A61M 25/0662; A61M 2025/0681

(57) ABSTRACT

Systems and devices for accessing an inner space between two layers of tissues are provided. A system can include a helical coil within a catheter delivery system. The helical coil can be delivered to the treatment site at which point it can be turned to penetrate and traverse tissue to reach an inner space. The system can further comprise a gas supply system for providing gas to help displace one or both tissue layers to create more access space within the inner space. A tool, prosthetic, or other device can be transferred via the catheter system into the inner space to perform a procedure therein.

### RELATED APPLICATIONS

This application is a continuation application of International Patent Application No. PCT/US2023/023878, filed May 30, 2023, which claims the benefit of both U.S. Provisional Application No. 63/347,466, filed May 31, 2022, and U.S. Provisional Application No. 63/481,099 filed Jan. 23, 2023, the entireties of which are hereby incorporated by reference.

### TECHNOLOGICAL FIELD

The disclosure is generally directed to systems and devices for providing a means to access space between layers of tissue.

### BACKGROUND

The pericardium, also referred to as the pericardial sac, is double-layered tissue that surrounds the heart and provides it protection. The outer layer of the pericardium is the fibrous pericardium formed of strong connective tissue and the inner layer is the serious pericardium formed of a serous membrane. Between the pericardium and the heart is the pericardial space, which a lubricous space that allows the heart to function without friction.

Heart failure is condition in which the heart muscle pumps a low ejection fraction of blood, resulting in poor blood circulation. While there are various causes of heart failure, the elastic pericardium can contribute to the low ejection fraction by exerting a compressive force on the heart myocardium. Thus, one way to increase to improve heart function is to relieve the compressive force provided by the pericardium.

### SUMMARY

A system for accessing an inner space between two tissues can comprise a helical coil within a catheter system. The helical coil is able to move along an axis in the distal direction such that it can be exposed out of the catheter system to perform a procedure. The helical coil is capable of turning such that the helical can penetrate and traverse a tissue. The system for accessing an inner space can further comprise a gas supply and/or vacuum. The gas supply can release gas within an inner space to displace one or both tissues to create more space therein. A vacuum can help hold a tissue and provide a resistance force for the helical coil to penetrate and traverse a tissue. A vacuum can also be utilized to remove the gas released within the inner space. A tool, prosthetic, or other device can be entered into the inner space to perform a procedure or treatment.

In one implementation, a transcatheter system comprises an outer sheath, an inner sheath, and a helical coil in connection with the inner sheath. The helical coil and the inner sheath are within the outer sheath. The helical coil and the inner sheath are capable of bidirectionally moving along an axis, independently of the outer sheath.

In some aspects, alone or in combination with any of the previous aspects, the system further comprises a nosecone connected to the outer sheath at the distal end. The nosecone is capable of opening to expose the helical coil.

In some aspects, alone or in combination with any of the previous aspects, the nosecone comprises a sensor for detecting a change in local environment.

In some aspects, alone or in combination with any of the previous aspects, the helical coil comprises at least one-half revolutions.

In some aspects, alone or in combination with any of the previous aspects, the helical coil comprises an axial distance of at least 2 mm.

In some aspects, alone or in combination with any of the previous aspects, the helical coil comprises a sensor at the distal end of the coil for detecting a change in local environment.

In some aspects, alone or in combination with any of the previous aspects, the system further comprises a gas supply system within the outer sheath. The gas supply system comprises a release port at a distal end, a gas supply at a proximal end, and a connection line therebetween.

In some aspects, alone or in combination with any of the previous aspects, the helical coil comprises a lumen. The connection line comprises the lumen of the helical coil. The release port is located at or proximal to the distal tip of the helical coil.

In some aspects, alone or in combination with any of the previous aspects, the system further comprises a vacuum.

In some aspects, the gas supply system is also a vacuum system.

In some aspects, alone or in combination with any of the previous aspects, the system further comprises a tool, a prosthetic, or a medicinal delivery device. The tool, the prosthetic, or the medicinal delivery device is within the outer sheath and is capable of bidirectionally moving along an axis, independently of the outer sheath.

In some aspects, alone or in combination with any of the previous aspects, the tool, the prosthetic, or the medicinal delivery device is capable of traversing through an inner circumference of the helical coil when traversed therethrough when moved bidirectionally along the axis.

In some aspects, alone or in combination with any of the previous aspects, the system further comprises a control system at a proximal end of the transcatheter system. The control system is capable of distally advancing and proximally retrieving the outer sheath.

In some aspects, alone or in combination with any of the previous aspects, the control system is further capable of distally advancing and proximally retrieving the helical coil.

In some aspects, alone or in combination with any of the previous aspects, the control system is further capable of rotating the helical coil.

In some aspects, alone or in combination with any of the previous aspects, the helical coil is sterilized.

In one implementation, a method comprises traversing a helical coil through a recipient to a procedural site via a transcatheter system. The transcatheter system comprises an outer sheath, an inner sheath, and a helical coil in connection with the inner sheath. The helical coil and the inner sheath are within the outer sheath. When the helical coil comes within proximity of the procedural site, the method further comprises distally advancing and exposing the helical coil out of the outer sheath such that the helical coil contacts a surface of a first tissue. The method further comprises rotating the helical coil such that the helical penetrates and traverses the first tissue. The helical coil is rotated until it traverses completely through the first tissue and is within an inner space between the first tissue and a second tissue.

In some aspects, alone or in combination with any of the previous aspects, the complete traversal through the first tissue and into the inner space is determined or is approximated by a visualization method.

In some aspects, alone or in combination with any of the previous aspects, the complete traversal through the first tissue and into the inner space is determined or is approximated by the amount of rotation of the helical coil.

In some aspects, alone or in combination with any of the previous aspects, the complete traversal through the first tissue and into the inner space is determined or is approximated by a sensor on the helical coil capable of detecting a change in local environment.

In some aspects, alone or in combination with any of the previous aspects, the method further comprises creating more access space within the inner space by displacing one of or both of the first tissue and the second tissue.

In some aspects, alone or in combination with any of the previous aspects, the first tissue is displaced by pulling the helical coil back in the proximal direction.

In some aspects, alone or in combination with any of the previous aspects, the transcatheter system further comprises a gas supply system within the outer sheath. The gas supply system comprises a release port at a distal end, a gas supply at a proximal end, and a connection line therebetween. The more access space within the inner space is created by releasing gas within the inner space to displace one of or both of the first tissue and the second tissue.

In some aspects, alone or in combination with any of the previous aspects, the transcatheter system further comprises a vacuum system within the outer sheath, and the method further comprises removing the gas within the inner space via the vacuum system.

In some aspects, alone or in combination with any of the previous aspects, the transcatheter system further comprises a vacuum system within the outer sheath, and prior to the rotation of the helical coil, the method further comprises grasping the first tissue via the vacuum.

In some aspects, alone or in combination with any of the previous aspects, the transcatheter system further comprises a tool within the outer sheath and the method further comprises distally advancing and exposing the tool out of the outer sheath, traversing the tool through the first tissue into the inner space, and performing a procedure with the tool within the inner space.

In some aspects, the transcatheter system further comprises a prosthetic within the outer sheath, and the method further comprises distally advancing and exposing the prosthetic out of the outer sheath, traversing the prosthetic through the first tissue into the inner space, and installing the prosthetic within the inner space.

In some aspects, alone or in combination with any of the previous aspects, the transcatheter system further comprises a medicinal delivery device within the outer sheath, and the method further comprises In some aspects, distally advancing and exposing the medicinal delivery device out of the outer sheath, traversing the medicinal delivery device through the first tissue into the inner space, and releasing a medicine from the medicinal delivery device within the inner space.

In some aspects, alone or in combination with any of the previous aspects, the transcatheter system further comprises a nosecone attached to the distal end of the outer sheath, and prior to exposing the helical coil, the method further comprises opening the nosecone.

In some aspects, alone or in combination with any of the previous aspects, the nosecone is opened by the distal advancement of the helical coil.

In some aspects, alone or in combination with any of the previous aspects, the first tissue is a pericardium and the second tissue is a myocardium.

In some aspects, alone or in combination with any of the previous aspects, the pericardium is reached via a subxiphoid approach.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIGS. 1A, 1B, and 1C provide views of an exemplary system for accessing and creating space between two tissue layers, in which the system is shown as being provided within a catheter. FIG. 1A is peripheral view. FIG. 1B is a cross-sectional view. FIG. 1C is an exploded view.

FIG. 2 provides an example of an exemplary catheter system opened to expose the helical coil and gas supply system for accessing and creating space between two tissue layers.

FIGS. 3A to 3F provide an exemplary use of the exemplary catheter for accessing and creating space between two tissue layers.

### DETAILED DESCRIPTION

The current disclosure details systems and devices for accessing and creating space between two tissues. The systems and devices can utilize a catheter design for minimal invasiveness within the body. Accordingly, systems and devices can comprise a catheter that provides a means for translocating tools for accessing and creating space between two tissue layers. Further, the catheter can provide a means for translocating tools to the space between the two layers to perform a procedure therein.

Systems and devices are directed to catheter systems that further comprise a helical coil, which can be utilized to traverse the outer tissue of the two tissues. Once traversed, a gas supply system can be utilized to supply a gas to the space between the two tissues. The gas can be utilized to displace one or both of the tissues to create a space therebetween. In some instances, the helical coil contains a hollowed lumen and release port, which can be connected to a gas supply in order to supply the gas to the space between the two tissues. The catheter system can further comprise a tool, a prosthetic, medication, or any other device or composition to be delivered into the inner space between the two tissues. Exemplary tools include a cutting tool, an electrophysiology tool, a suturing system, a sensor, a visualization enhancing tool (e.g., camera or radiopaque device), or any other tool that can fit within the catheter and would be desired by the clinician to be delivered to the inner space between two tissues. Exemplary prosthetics include a stent, a valve, a patch, sutures, staples, an implantable sensor, or any other prosthetic that can fit within the catheter and would be desired by the clinician to be delivered to the inner space between two tissues. Exemplary medications include antibiotics, analgesics, wound healing medication, anti-cancer medication, immunostimulant, immunosuppressant, or any other medication that can be delivered via catheterization and would be desired by the clinician to be delivered to the inner space.

The described systems, devices, and methods should not be construed as limiting in any way. Instead, the present disclosure is directed toward all novel and nonobvious features and aspects of the various disclosed systems and devices, alone and in various combinations and sub-combinations with one another. The disclosed systems, devices, and methods are not limited to any specific aspect, feature, or combination thereof, nor do the disclosed systems, devices, and methods require that any one or more specific advantages be present or problems be solved.

Various examples of space accessing systems and components thereof are disclosed herein, and any combination of these examples can be made unless specifically excluded. For example, a helical coil device can be used with any catheter system or any gas supply system, even if a specific combination is not explicitly described. Likewise, the different constructions and features of space accessing systems can be mixed and matched, such as by combining any tool for entering into the inner space, any system for providing displacement gas, and any device, or composition to be utilized within the space, even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

The terms “proximal” and “distal” as used throughout the description relate to a catheter system axis, in which the end where the procedure is performed is the distal end and the opposite end where the catheter system is controlled is the proximal end. Accordingly, the distal end of the catheter system is the leading end that first traverses into the body and first reaches the procedure site. Conversely, the proximal end of the catheter system is the portion that remains extracorporeal. Likewise, a distal movement along the catheter axis would be movement of a component in a direction towards a site of procedure and a proximal movement along the catheter axis would be movement of a component in an opposite direction. Although these terms have a relationship with a site of procedure, it is to be understood that these terms are used for reference and the site of procedure does not need to be present when interpreting the components or movements of the devices and systems described herein.

Various systems and devices for accessing an inner space between two tissues are utilized for the purpose of performing a procedure within a recipient. Recipients

include (but are not limited to) patients, animal models, cadavers, or anthropomorphic phantoms. Accordingly, in addition to methods of treating patients, the systems and devices can be utilized in training or other practice procedures upon animal models, cadavers, or anthropomorphic phantoms.

The described systems and devices can be sterilized, which can be performed using gamma irradiation, gas plasma, aldehydes, ethylene oxide, and/or e-beam. The systems or devices can be further treated with a formaldehyde bioburden reduction process. After preparation, the systems and devices can be stored within a container, which can be hermetically sealed or otherwise kept sterile.

### Systems and Devices for Accessing Inner Space Between Two Tissues

Systems and devices for accessing an inner space between two tissues can comprise a catheter system such that it can be utilized within minimally invasive procedures. The systems and devices can comprise a means for traversing the outer layer into the inner space between the two layers. The systems and devices can further comprise a gas-supply system to provide a gas to the inner space to displace one or both tissues to create an accessible space between the two tissues. The systems and devices can further comprise a tool, a prosthetic, a medication, or other device or composition to be transported to the inner space.

The systems and devices for accessing an inner space can be utilized on any two tissues in which an inner space can be accessed. In many instances, the systems and devices are utilized to access an inner space between connective tissue and muscular tissue. One such example an inner space to be accessed is the pericardial space between the pericardium and the myocardium. The pericardium is a connective tissue that surrounds the muscular heart tissue. Any catheter approach can be utilized to reach the two tissues and inner space. For example, a subxiphoid approach can be utilized to access the pericardial space.

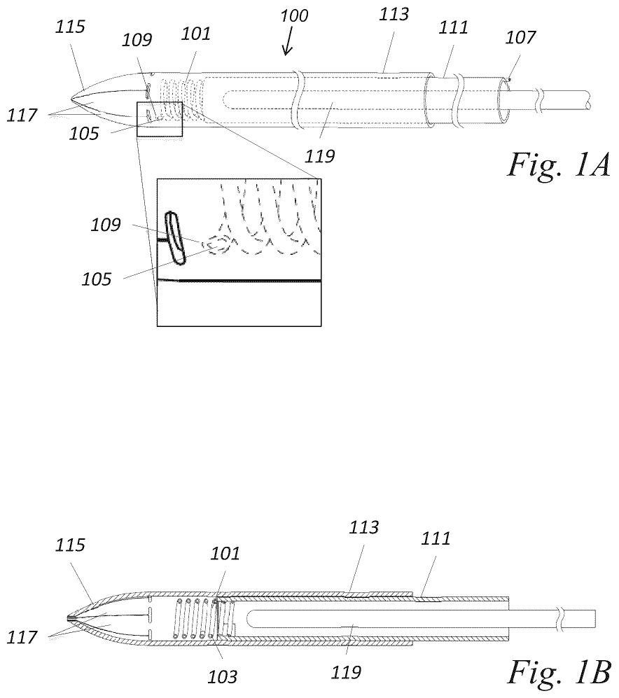

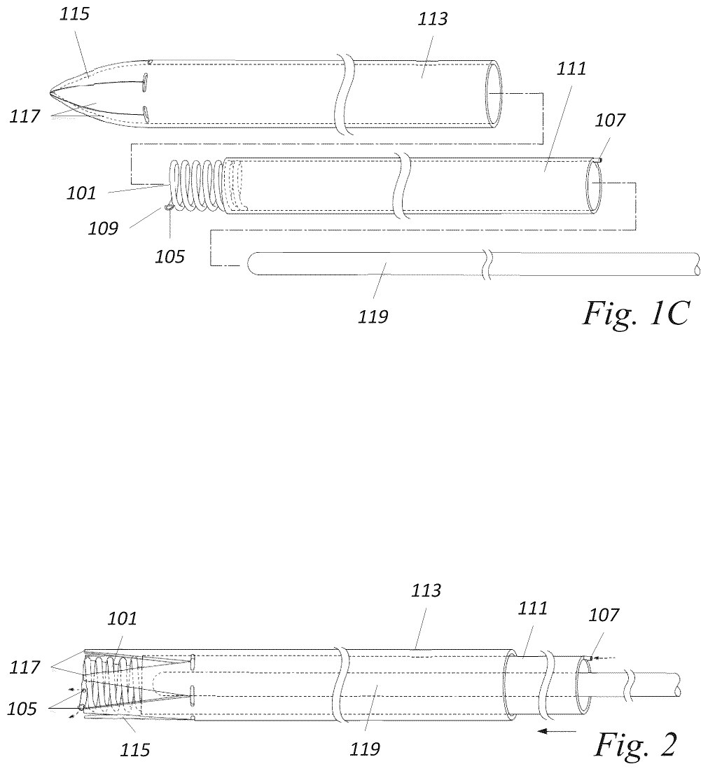

Provided in FIGS. 1A, 1B, and 1C is an example of a system for accessing the inner space between two tissues, the system is utilized with a transcatheter system. The system comprises a helical coil 101 and a gas supply system. As shown, the gas supply system comprises an inner lumen 103 within helical coil 101, a set of release ports 105, a gas supply (not shown), and a connection line 107 for connecting the gas supply with the inner lumen and release port. Helical coil 101 provides the means for traversing the outer tissue of two tissue such that the release port 105 can enter into the inner space between two tissues. The gas supply system provides the means for providing gas into the inner space to displace one or both tissues to create accessible space between the two tissues. Helical coil 101 can comprise a tip 109 that is pointed or otherwise capable of puncturing or traversing through tissue.

Helical coil 101 can be advanced along an axis in the distal and/or proximal direction. Helical coil can be advanced by any capable means within a transcatheter system. As shown in FIGS. 1A to 1C, an inner sheath 111 is in connection with the proximal end of helical coil 101. Advancement of inner sheath 111 pushes helical coil 101 in the distal direction and retrieval of the inner sheath pulls helical coil in the proximal direction.

Helical coil 101 can comprise a number of revolutions; a revolution being one full circle of a helical turn. Generally, at least one half of a revolution can be provided such that coil can be twisted through an outer tissue into an inner space. In some instances, the number of revolutions corresponds to the axial distance per revolution and the tissue depth to be traversed. For example, the thickness human pericardial tissue is approximately between 2 mm and 3 mm. In some instances, an axial distance per revolution can be 1 mm and thus at least 2 or 3 turns are provided such that the helical coil can traverse through the pericardial thickness to reach the inner pericardial space. Further, having a precise axial distance of per revolution can allow the clinician to approximate the distance traversed through the tissue. For example, when the axial distance per revolution is 1 mm, the clinician can know that one 1 to 1.5 turns reaches approximately half way through the pericardium and that 2 to 3 turns reaches approximately the inner space. Based on the foregoing, in various instances, the helical coil comprises at least one-half revolutions and further comprises a number of revolutions with an axial distance between each revolution such that the number of revolutions allows the helical coil to completely traverse the thickness of the tissue to be traversed.

In various implementations, the helical coil has between 0.5 and 15 revolutions. In some particular implantations, the helical coil has 0.5. revolutions, 1.0 revolutions, 1.5 revolutions, 2.0 revolutions, 2.5 revolutions, 3.0 revolutions, 3.5 revolutions, 4.0 revolutions, 4.5 revolutions, 5.0 revolutions. 5.5 revolutions, 6.0 revolutions, 6.5. revolutions, 7.0 revolutions, 7.5 revolutions, 8.0 revolutions, 8.5 revolutions, 9.0 revolutions, 9.5 revolutions, 10.0 revolutions, 10.5 revolutions, 11.0 revolutions. 11.5 revolutions, 12.0 revolutions, 12.5 revolutions, 13.0 revolutions, 13.5 revolutions, 14.0 revolutions. 14.5 revolutions, or 15.0 revolutions.

In various implementations, the helical coil has an axial distance of 0.5 mm and 10 mm between each revolution. In some particular implantations, the helical coil has an axial distance of 0.5 mm between each revolution, an axial distance of 1.0 mm between each revolution, an axial distance of 1.5 mm between each revolution, an axial distance of 2.0 mm between each revolution, an axial distance of 2.5 mm between each revolution, an axial distance of 3.0 mm between each revolution, an axial distance of 3.5 mm between each revolution, an axial distance of 4.0 mm between each revolution, an axial distance of 5.5 mm between each revolution, an axial distance of 5.0 mm between each revolution, an axial distance of 6.5 mm between each revolution, an axial distance of 6.0 mm between each revolution, an axial distance of 7.5 mm between each revolution, an axial distance of 7.0 mm between each revolution, an axial distance of 8.5 mm between each revolution, an axial distance of 8.0 mm between each revolution, an axial distance of 8.5 mm between each revolution, an axial distance of 9.0 mm between each revolution, an axial distance of 9.5 mm between each revolution, or an axial distance of 10.0 mm between each revolution.

The diameter of helical coil 101 can vary. Generally, the outer diameter is less than an outer catheter diameter such and maintains a low profile when traversing the body. In some instances, the inner diameter is large enough such that it has space to allow a tool, a prosthetic, or another device to pass therethrough (as described in greater detail below).

In various implementations, the helical coil has an outer diameter between about 2 mm and about 5 mm. In some particular implementations, the helical coil has an outer diameter of 2.0 mm, an outer diameter of 2.5 mm, an outer diameter of 3.0 mm, an outer diameter of 3.5 mm, an outer diameter of 4.0 mm, an outer diameter of 4.5 mm, or an outer diameter of 5.0 mm. In various implementations, the helical coil has an inner diameter between about 1 mm and about 4 mm. In some particular implementations, the helical coil has an inner diameter of 1.0, an inner diameter of 1.5 mm, an inner diameter of 2.0 mm, an inner diameter of 2.5 mm, an inner diameter of 3.0 mm, an inner diameter of 3.5 mm, or an inner diameter of 4.0 mm.

The axial length of helical coil 101 can vary. Generally, the axial length is long enough to completely traverse the tissue but short enough to easily transit through the body in the minimally invasive procedure. As described in an earlier example, the thickness human pericardial tissue is approximately between 2 mm and 3 mm, however some tissues can be approximately 1 mm. To assure easy transit through the body, the axial length should not be longer than 20 mm.

In various implementations, the helical coil has an axial length between about 1 mm and about 20 mm. In some particular implementations, the helical coil has an axial length of 1.0 mm, an axial length of 1.5 mm, an axial length of 2.0 mm, an axial length of 2.5 mm, an axial length of 3.0 mm, an axial length of 3.5 mm, an axial length of 4.0 mm, an axial length of 4.5 mm, an axial length of 5.0 mm, an axial length of 6.0 mm, an axial length of 6.5 mm, an axial length of 7.0 mm, an axial length of 7.5 mm, an axial length of 8.0 mm, an axial length of 8.5 mm, an axial length of 9.0 mm, an axial length of 9.5 mm, an axial length of 10.0 mm, an axial length of 11.0 mm, an axial length of 11.5 mm, an axial length of 12.0 mm, an axial length of 12.5 mm, an axial length of 13.0 mm, an axial length of 13.5 mm, an axial length of 14.0 mm, an axial length of 14.5 mm, an axial length of 15.0 mm, an axial length of 16.0 mm, an axial length of 16.5 mm, an axial length of 17.0 mm, an axial length of 17.5 mm, an axial length of 18.0 mm, an axial length of 18.5 mm, an axial length of 19.0 mm, an axial length of 19.5 mm, or an axial length of 20.0 mm.

In some implementations, the helical coil includes a sensor at the distal end of the coil. In some implementations, the sensor detects a change in local environment, such as (for example) contacting the first tissue, traversing the first tissue, exiting the first tissue, entering into the inner space between tissues, contacting the second tissue, and/or traversing the second tissue. In some embodiments, the sensor detects electrical impedance. The sensor can transmit data via connection through the delivery transcatheter system or via radio-frequency transmission (e.g., RFID).

Several implementations of a system for accessing and creating space between two tissues utilizes a gas supply system to provide gas between the two tissues to displace one or both tissues via the gas. As shown in FIGS. 1A to 1C, a gas supply system can comprise an inner lumen 103 within helical coil 101 and a release port 105 on the coil. The gas supply system further contains a connection line 107 to connect release port 105 to a gas supply at a proximal end of the connection line. A connection line can be any sort of tubing or piping capable of providing a sealed connection between the gas supply and the release port. In many implementations, the connection line is composed of a flexible material such that it can bend and flex with the transcatheter delivery system as the delivery system traverses through the body to the site of the two tissues to be accessed.

Although FIGS. 1A to 1C show release port 105 at the tip of helical coil 101, it should be understood that the release port can be provided anywhere along the coil. In several implementations, the release port is provided near the distal end of the helical coil such that when the distal end of the coil reaches the space between two tissues the gas from the gas supply can be released within that space.

In some implementations, a gas supply system utilizes the helical coil to supply the gas to the inner space, as shown in FIGS. 1A to 1C. It should be understood, however, that the gas supply system can be completely or partially independent of the helical coil. Accordingly, in some implementations, the gas supply incorporates a release port and/or a connection line that is independent of the helical coil. In some implementations, the gas supply release port is capable of being moved along an axis in the distal direction and/or in the proximal direction. In some implementations, the gas supply release port is capable of being pushed into inner space between the two tissues such that gas can be released therein.

Any gas capable of being released within human can be utilized. In some implementations, the gas is a biocompatible gas, such as (for example) carbon dioxide. In some implementations, the gas is an inert gas, such as (for example) nitrogen.

In some implementations, a vacuum system is provided in a system for accessing and creating space between two tissues. The vacuum system can utilize the same ports, connection lines, and/or machinery as the gas supply system or the vacuum system can utilize its a separate set of one or more ports, a separate set of one or more connection lines, or separate machinery as the gas supply system. In several implementations, the vacuum system is utilized to remove gas (or other fluids or debris) from the inner space between the two tissues. In some implementations, the vacuum system is utilized to grasp tissue by sucking it inward, which can provide the helical coil a resistance force to penetrate and/or traverse tissue.

In some implementations, system for accessing the inner space between two tissues does not include a gas supply system or a vacuum system. In these implementations, the helical coil is utilized penetrate and traverse the outer tissue to reach the inner space between the two tissues. To create more space within the inner space, the helical coil can be pulled back in the proximal direction such that the outer tissue is pulled away from the inner tissue.

The system for accessing space between two tissues can be delivered through the body via a catheter and transcatheter system. As shown in FIGS. 1A to 1C, the catheter system comprises an outer sheath 113 surrounds helical coil 101 and extends along an axis.

At the distal end of outer sheath 113 is a nosecone 115 angled to facilitate traversing through the body. The nosecone of the catheter system is capable of opening to allow the inner contents (e.g., helical coil) to be advanced therethrough. Provided in FIG. 2 is an exemplary depiction of the catheter system in which nosecone 115 has been opened to allow helical coil 101 to advance proximally out of outer sheath 113. Nosecone 115 comprises a number of segments 117 with a pointed angle at the distal end such that the segments can form a conical nosecone when closed (FIG. 1A) and further capable of opening as helical coil 101 is advanced distally (FIG. 2).

In some implementations, the nosecone includes a sensor at the distal end of the cone. In some implementations, the sensor detects a change in local environment, such as (for example) traversing through the body, and/or contacting the tissue. In some embodiments, the sensor detects electrical impedance. The sensor can transmit data via connection through the delivery transcatheter system or via radio-frequency transmission (e.g., RFID).

The catheter system extends proximally to a control system, as is appreciated in the art of transcatheter procedures. A clinician can utilize the control system to advance the transcatheter system through the body to the site where the procedure is to be performed. Further, the control system can advance and retract the helical coil in the axial and distal direction in reference to the outer sheath. The control system can be utilized to turn the helical coil such that it can penetrated and traverse through tissue. The control system can further comprise a means for regulating the gas supply and/or vacuum such that gas can be transferred and released from the distal release port and/or vacuumed from the distal port. The control system can further comprise a means for controlling a set of one or more optional tools to be utilized upon accessing an inner space between two tissues.

In some implementations, a system for accessing an inner space between two tissue layers further comprises a tool, a prosthetic, a device for delivery of medication, or any other device or composition to be delivered into the inner space between the two tissues. As shown in FIGS. 1A to 1C, within the central lumen of inner sheath 111 is a tool 119 capable of being moved along the axis in a distal direction or in a proximal direction. In many implementations, the tool is appropriately sized to fit within the inner lumen of the inner sheath and/or the inner circumference of the helical coil such that it moves distally and/or proximally in reference to the helical coil. Accordingly, in some instances, the tool, the prosthetic, or the medication delivery device is pushed distally beyond the helical coil such that it can enter within the inner space between the two tissues. In some instances, the tool, the prosthetic, or the medication delivery device includes a sharp edge at the distal end such that it can cut through tissue in order to reach the inner space.

Exemplary tools include a cutting tool, an electrophysiology tool, a suturing system, a sensor, a visualization enhancing tool (e.g., camera or radiopaque device), or any other tool that can fit within the catheter and would be desired by the clinician to be delivered to the inner space between two tissues. Exemplary prosthetics include a stent, a valve, a patch, sutures, staples, an implantable sensor, or any other prosthetic that can fit within the catheter and would be desired by the clinician to be delivered to the inner space between two tissues. Exemplary medications include antibiotics, analgesics, wound healing medication, anti-cancer medication, immunostimulant, immunosuppressant, or any other medication that can be delivered via catheterization and would be desired by the clinician to be delivered to the inner space.

While a specific configuration of a system for accessing the inner space between two tissues is described above with reference to FIGS. 1A to 1C, it should be readily appreciated that various configurations of system for accessing the inner space between two tissues can be implemented in any of a variety of combinations of components. Accordingly, the specific configuration of a system for accessing the inner space between two tissues described herein should be understood as not to be limited to any specific configuration, but instead can be implemented in any configuration capable of accessing an inner space between two tissues.

### Methods of Use

The exemplary systems for accessing the inner space between two tissues of the disclosure can be utilized in a catheter system. In many instances, an incision is made the body and the catheter system is inserted therein in order to reach the site of the procedure. In instances in which the pericardial space is to be accessed, a subxiphoid approach can be utilized. Once the catheter system reaches the site procedure, the catheter can open up such that the helical coil can be advanced distally to come into contact with the tissue. At that point, the helical coil can be utilized to traverse the tissue to reach the inner space. In some instances, the method is view contemporaneously via a visualization method, such as (for example) sonography.

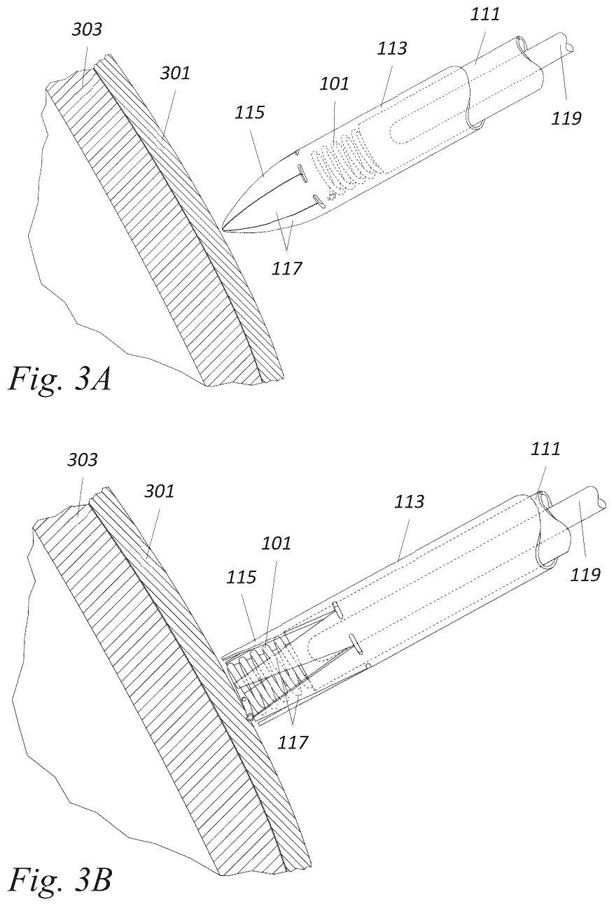

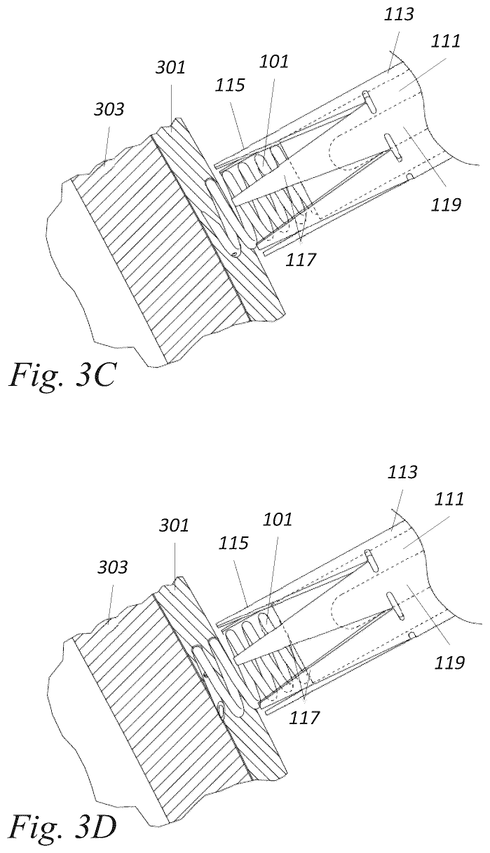

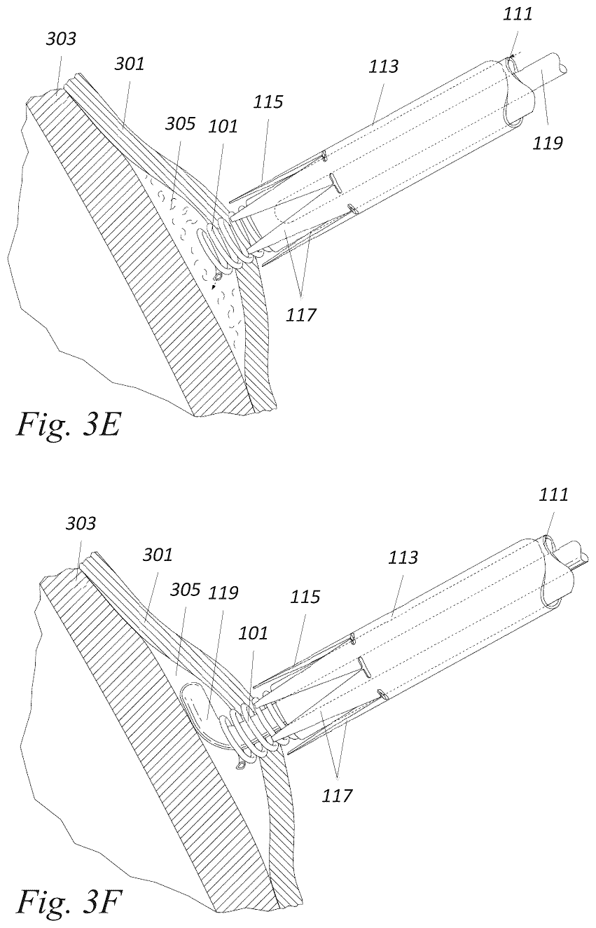

Provided in FIGS. 3A to 3F is an exemplary method of utilizing a system for accessing the inner space between two tissues. FIG. 3A depicts the catheter system approaching a first tissue layer 301. Nosecone 115 can be advanced distally until it contacts the surface of tissue layer 301. In some implementations, the nosecone can incorporate a sensor at the distal end such that the sensor can sense contact with the tissue layer.

Upon contact with the surface of tissue layer 301, segments 117 of nosecone 115 can be opened outwardly allowing the distal advancement of helical coil 101 (FIG. 3B). In some implementations, distally advancing helical coil 101 pushed segments 117 open. Helical coil 101 can advance distally until it contacts the surface of tissue layer 301, at which point the helical coil can be turned such that the distal tip of the helical coil penetrates and traverses the tissue (FIG. 3C).

Helical coil 101 continues to turn until it comes in contact with the surface of a second tissue 303 (FIG. 3D). In some instances, contact of helical coil 101 with the surface of tissue 303 is determined by a sensor (e.g., electrical impedance sensor). In some instances, contact of helical coil 101 with the surface of tissue 303 is determined by knowing the thickness of the first tissue and the number of turns it would take to completely traverse. In some instances, contact of helical coil 101 with the surface of tissue 303 is determined by a visualization method (e.g., sonography).

When helical 101 contacts tissue 303, a gas supply system releases gas into inner space 305 resulting in a displacement or lifting of tissue 301 (FIG. 3E). Alternatively, or in addition to release of gas, the catheter system can be moved in the proximal direction to lift tissue 301. The displacement or lifting of tissue 301 creates a more access space within the inner space 305, at which point tool 119 can enter within the inner space (FIG. 3F). In some instances, tool 119 includes a sharp edge at the distal end to pierce through tissue 301 to reach inner space 305.

We claim:

1. A system for accessing an inner space between two regions of tissue, comprising:
a transcatheter system, wherein the transcatheter system comprises an outer sheath, an inner sheath, and a helical coil in connection with the inner sheath, wherein the helical coil and the inner sheath are within the outer sheath, and wherein the helical coil and the inner sheath are capable of bidirectionally moving along an axis, independently of the outer sheath.

2. The system of claim 1 further comprising a nosecone connected to the outer sheath at a distal end, wherein the nosecone is adjustable for exposing the helical coil.

3. The system of claim 2, wherein the nosecone comprises a sensor for detecting a change in local environment.

4. The system of claim 1, wherein the helical coil comprises at least one-half of a revolution.

5. The system of claim 1, wherein the helical coil comprises an axial distance of at least 2 mm.

6. The system of claim 1, wherein the helical coil comprises a sensor at a distal end of the helical coil.

7. The system of claim 1, further comprising a gas supply system within the outer sheath, wherein the gas supply system comprises a release port at a distal end, a gas supply at a proximal end, and a connection line therebetween.

8. The system of claim 7, wherein the helical coil comprises a lumen, and wherein the release port is positioned in the lumen at or proximal to a distal tip of the helical coil.

9. The system of claim 1, further comprising a vacuum system.

10. The system of claim 1, further comprising a tool, a prosthetic, or a medicinal delivery device, wherein the tool, the prosthetic, or the medicinal delivery device is within the outer sheath and is capable of bidirectionally moving along an axis, independently of the outer sheath, wherein the tool, the prosthetic, or the medicinal delivery device is capable of traversing through an inner circumference of the helical coil when traversed therethrough when moved bidirectionally along the axis.

11. The system of claim 1, further comprising a control system at a proximal end of the transcatheter system, wherein the control system is capable of distally advancing and proximally retrieving the outer sheath, wherein the control system is further capable of distally advancing and proximally retrieving the helical coil and/or wherein the control system is further capable of rotating the helical coil.

12. A transcatheter device, comprising:
an outer sheath;
an inner sheath;
a helical coil in connection with the inner sheath; and
a gas supply system comprising a release port at a distal end, a gas supply at a proximal end, and a connection line therebetween,
wherein the helical coil, the inner sheath, and gas supply system are within the outer sheath,
wherein the helical coil and the inner sheath are bidirectionally movable along an axis of the transcatheter device, independently of the outer sheath, and
wherein the helical coil comprises a lumen, and wherein the release port is located in the lumen at or proximal to a distal tip of the helical coil.

13. The device of claim 12, further comprising a nosecone connected to the outer sheath at a distal end, wherein the nosecone opens to expose the helical coil.

14. The device of claim 13, wherein the nosecone comprises a sensor.

15. The device of claim 12, wherein the helical coil comprises at least one revolution or wherein the helical coil comprises an axial distance of at least 2 mm.

16. The device of claim 12, wherein the helical coil comprises a sensor at a distal end of the helical coil.

17. The device of claim 12, wherein the helical coil comprises a lumen.

18. The device of claim 12, further comprising at least one of a tool, a prosthetic, or a medicinal delivery device, wherein the tool, the prosthetic, or the medicinal delivery device is within the outer sheath and is bidirectionally movable along the axis of the transcatheter device, independently of the outer sheath, wherein the tool, the prosthetic, or the medicinal delivery device is bidirectionally traversable through an inner circumference of the helical coil, and/or wherein the control system is capable of distally advancing and proximally retrieving the outer sheath.

19. The device of claim 18, wherein the control system distally advances and proximally retrieves the helical coil and/or rotates the helical coil.

20. The device of claim 12, wherein the helical coil is sterilized.
